(19) United States
(12) Patent Application Publication
(10) Pub. No.: US 2014/0199722 A1
(43) Pub. Date: Jul. 17, 2014

(54) METHODS FOR DETERMINING WHETHER A CERVICAL CELLULAR SAMPLE SHOULD BE TESTED FOR CERVICAL CANCER, AND DEVICES AND KITS FOR PRACTICING THE SAME

(71) Applicant: INCELLDX, INC., Menlo Park, CA (US)

(72) Inventor: Bruce K. Patterson, Palo Alto, CA (US)

(21) Appl. No.: 14/153,884

(22) Filed: Jan. 13, 2014

Related U.S. Application Data
(60) Provisional application No. 61/752,237, filed on Jan. 14, 2013.

(51) Int. Cl. G01N 33/50

(52) U.S. Cl. CPC G01N 33/5091
USPC 435/29; 435/287.1

(57) ABSTRACT

Methods for determining whether a cervical cellular sample should be tested for cervical cancer are provided. Aspects of the methods include obtaining cytological data from a cervical cellular sample, and determining whether the cellular sample should be tested for cervical cancer based on the cytological data. Also provided are devices and kits that find use in practicing the methods. The methods, devices and kits find use in a variety of applications, including cervical cancer prescreening.

### CROSS-REFERENCE TO RELATED APPLICATIONS

Pursuant to 35 U.S.C. §119(e), this application claims priority to U.S. Provisional Application Ser. No. 61/752,237 filed on Jan. 14, 2013, the disclosure of which is herein incorporated by reference.

### INTRODUCTION

Cervical cancer is a malignant neoplasm arising from cells originating in the cervix uteri. Cervical cancer is the second most common cause of cancer-related mortality in women worldwide. Epidemiological and laboratory studies suggest a key role for human papillomavirus (HPV) in cervical carcinogenesis (Walboomers, J. M. et al. (1999) J. Pathol. 189:12-19; Zur, H. H. (2002) Nat. Rev. Cancer 2:342-350). Importantly, however, HPV infection alone is not sufficient for cervical carcinogenesis, and additional steps occur over years or decades following initial infection. Most HPV infections resolve spontaneously, but if an oncogenic (high risk) HPV infection persists, there may be progression to a high grade cervical dysplasia or cervical cancer. (Nobbenhuis, M. A. et al. (2001) Lancet 358: 1782-1783). High risk HPVs include HPV-16, 18, 31, 33, 35, 39, 45, 51, 52, 56, 58, 59, 66, and 68, with HPV-16 and 18 accounting for up to 70% of cervical cancers worldwide.

Screening for cervical cancer can be performed using the Papanicolaou test (PAP test) and/or Human papillomavirus (HPV) testing. The Papanicolaou (Pap) smear has become the most commonly used method to screen for cervical dysplasia. It has been a success and the incidence of cervical cancer has been dramatically reduced. However, cytology screening programs have limitations, especially limited sensitivity, estimated at only 51% (Nanda K. et al. (2000) Ann. Intern. Med. 132:810-819), and repeated tests are therefore necessary. In addition, a high-quality cytology screening program requires highly-trained personnel. Furthermore, although cytological screening programs have reduced the incidence of squamous cervical cancer (SCC), the incidence of cervical adenocarcinoma (AC) has continued to increase. The reason for this is unclear, but it may, in part, be due to difficulties detecting the precursor form of AC using conventional screening methods. (Bray, F. B. et al. (2005) Cancer Epidemiol. Biomarkers Prev. 14:2191-2199).

HPV DNA testing can be more sensitive than cytologic testing in detecting high-grade cervical dysplasia. However, HPV testing often has lower specificity than cytologic testing since most HPV infections are transient in nature. (Koliopoulous, G. M. et al. (2007) Gynecol. Oncol. 104:232-246).

HPV testing has been used as an adjunct to PAP testing for cervical cancer screening for over a decade. Some have proposed the use of HPV DNA testing to triage HPV DNA negative women to longer screening intervals (3-5 years). Such combinatorial screening creates, in general, two distinct workflows that are solely carried out in clinical laboratories. This workflow prolongs the time required for physicians to decide on appropriate management or therapeutic options.

### SUMMARY

Methods for determining whether a cervical cellular sample should be tested for cervical cancer are provided. Aspects of the methods include obtaining cytological data from a cervical cellular sample, and determining whether the cellular sample should be tested for cervical cancer, based on the cytological data. Also provided are devices and kits that find use in practicing the methods. The methods, devices and kits find use in a variety of applications, including cervical cancer prescreening applications.

### BRIEF DESCRIPTION OF THE FIGURES

FIG. 1 provides a graph showing the mean corpuscular volume of cervical squamous cells from various cytological categories. Cytological categories shown are: 1) negative for intraepithelial lesion or malignancy (NILM) cells: 2) atypical squamous cells of undetermined significance (ASCUS) cells that are either positive or negative for the HPV protein E6; and 3) low-grade squamous intraephithelial lesion (LSIL) cells that that are either positive or negative for the HPV protein E6.

FIGS. 2 A and 2B provide scatter plot graphs showing the relationship of post G₁ percentage data and total percentage of HPV protein E6 of cervical cellular samples that are either normal (NILM, FIG. 2A) or have abnormal cytology (FIG. 2B).

FIG. 3 illustrates identification of abnormal cells in a cervical cellular sample by nuclear to cytoplasmic ratio analysis. Increased N/C ratios are indicative of abnormal cells, high grad squamous intraepithelial lesion (HSIL).

### DETAILED DESCRIPTION

Methods for determining whether a cervical cellular sample should be tested for cervical cancer are provided. Aspects of the methods include obtaining cytological data from a cervical cellular sample, and determining whether the cellular sample should be tested for cervical cancer, based on the cytological data. Also provided are devices and kits that find use in practicing the methods. The methods, devices and kits find use in a variety of applications, including cervical cancer prescreening applications.

Before the present invention is described in greater detail, it is to be understood that methods, devices and kits provided herein are not limited to particular embodiments described, as such may, of course, vary. It is also to be understood that the terminology used herein is for the purpose of describing particular embodiments only, and is not intended to be limiting, since the scope of will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention, subject to any specifically excluded limit in the stated range. Where the stated range includes one or both of the limits, ranges excluding either or both of those included limits are also included.

Certain ranges are presented herein with numerical values being preceded by the term “about.” The term “about” is used herein to provide literal support for the exact number that it precedes, as well as a number that is near to or approximately the number that the term precedes. In determining whether a number is near to or approximately a specifically recited number, the near or approximating unrecited number may be a number which, in the context in which it is presented, provides the substantial equivalent of the specifically recited number.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, representative illustrative methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present invention is not entitled to antedate such publication by virtue of prior invention. Further, the dates of publication provided may be different from the actual publication dates which may need to be independently confirmed.

It is noted that, as used herein and in the appended claims, the singular forms “a”, “an”, and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

It is appreciated that certain features, which are, for clarity, described in the context of separate embodiments, may also be provided in combination in a single embodiment. Conversely, various features, which are, for brevity, described in the context of a single embodiment, may also be provided separately or in any suitable sub-combination. All combinations of the embodiments are specifically embraced by the present invention and are disclosed herein just as if each and every combination was individually and explicitly disclosed, to the extent that such combinations embrace operable processes and/or devices/systems/kits. In addition, all sub-combinations listed in the embodiments describing such variables are also specifically embraced by the present invention and are disclosed herein just as if each and every such sub-combination of chemical groups was individually and explicitly disclosed herein.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

In further describing embodiments, aspects of embodiments of the methods will be described first in greater detail. Next, embodiments of devices and kits that may be used in practicing methods provided herein are reviewed.

### Methods

As summarized above, methods for determining whether a cervical cellular sample should be tested for cervical cancer are provided. By determining whether a cervical cellular sample should be tested is meant assaying a cervical cellular sample to obtain data that correlates with cervical cancer and then determining whether the particular data that is obtained from the cervical cellular sample is indicative of cervical cancer. If the obtained data from the assayed sample is indicative of cervical cancer, then the sample is recommended to be tested for cervical cancer. Any data that correlates with cervical cancer may be used with the methods provided herein, including, but not limited to, cytological data, as described herein. Data from an assayed cervical cellular sample is determined to be indicative of cervical cancer if the data falls within a range or is above or below a threshold value that correlates with cervical cancer. In a particular embodiment of the method, the data is assayed and determined for testing for cervical cancer at the location of sample obtainment, e.g., in the same building, such as the same room. Such an embodiment allows for a convenient, first pass evaluation of whether a cervical cellular sample should undergo more cost-, labor-, and time-intensive tests for cervical cancer that are typically performed at a location different from where the cervical cellular sample is obtained. Aspects of the method are discussed in further detail below.

In certain embodiments, the methods include steps of assaying a cervical cellular sample to obtain cytological data; and determining from the cytological data whether the sample should be tested for cervical cancer. Thus, an aspect of methods described herein is a step of assaying the cellular sample to obtain cytological data. As used herein, “cytological data” refers to any property (data regarding morphology, formation, function, and development) of a cervical cell that can be used in the methods provided herein to determine whether a cervical sample should be tested for cervical cancer. In some embodiments, the assaying step comprises assaying one, two, three, four, five, six, seven, eight, nine, or ten or more different types of cytological data. In certain embodiments of the methods wherein more than one cytological data is assayed, each cytological data is assayed from a different aliquot of cervical cellular sample. In other embodiments, each cytological data is assayed from the same aliquot of cervical cellular sample.

Cytological data may include, for example, morphometric data. “Morphometric data” refers to any type of data from which cell morphology information (e.g., information about the size, shape and/or structure of a cell) may be derived. Morphometric data includes, but is not limited to, image data, forward scatter light data, side scatter light data, and combinations thereof. Image data refers to any data relating to captured images of cells from a cervical cellular sample as described herein. Image data can be obtained, for example, using a microscope (e.g., confocal microscope) with image capturing capabilities to capture microscopial images. See, e.g., Wang, Y. E. et al. (2010) PLoS Pathog 6(11): e1001186; White, F. H. et al. (1997) Histol. Histopathol. 12: 69-77. Forward scatter light data (FSC) and side scatter light data (SSC) are derived from the light scattering characteristics by cells in a cervical cellular sample that can be obtained using a flow cytometer. Forward scatter light data correlates with cell-surface area or size, whereas side scatter light data reflects the inner complexity of the cell (e.g., shape of the nucleus, amount and type of cytoplasmic granules, or membrane roughness). See, e.g., Rothe, G. (2009) Cellular Diagnostics. Basic Methods and Clinical Applications of Flow Cytometry, Basel, Karger, pp. 53-88.

Parameters of the morphometric data can include, but are not limited to, cell volume (e.g., mean corpuscular volume), nuclear area, cytoplasmic area, perimeter, texture, cell shape (e.g., round, elliptical, barbell-shaped, etc.), and ratios of these parameters (e.g., nuclear to cytoplasmic ratio). Several of these parameters are discussed in further detail below.

In certain embodiments, the cytological data is at least one of mean corpuscular volume (MCV) data, nuclear to cytoplasmic (NC) ratio data and post G₁ data. Thus, in certain embodiments, the cytological data is MCV data. In other embodiments, the cytological data is NC ratio data. In yet other embodiments, the cytological data is post G₁ data. In certain embodiments, the cytological data is two of mean corpuscular volume (MCV) data, nuclear to cytoplasmic (NC) ratio data and post G₁ data. Thus, in certain embodiments, the cytological data is MCV data and NC ratio data. In other embodiments, the cytological data is MCV data and post G₁ data. In yet other embodiments, the data is NC ratio data and post G₁ data. In other embodiments, the cytological data is MCV data, NC data and post G₁ data.

In certain embodiments, the cytological data is mean corpuscular volume data. As used herein, “mean corpuscular volume,” “mean cell volume,” and “MCV” all refer to the average cellular volume of the cervical cells within a cervical cellular sample described herein. Any suitable method can be used to determine mean cell volume. In certain embodiments, mean corpuscular volume is determined using an automated analyzer, such as a volume-sensitive automated cell counter. See, e.g., Moran, J. et al. (2001) Biochim Biophys Acta 1538: 313-320; Morales-Mulia, M. et al. (2000) Biochem Biophys Acta 1496: 252-260. Volume-sensitive automated cell counters can determine mean corpuscular volume, for example, through an electronic-based technique (e.g., electronic volume, based on the Coulter principle). Mean corpuscular volume can also be measured using protocols and apparatuses that measure refracted, diffracted or scattered light. See, e.g., Tzur, A. et al. (2011) PLoS ONE 6(1): e16053. In yet other embodiments, mean cell volume is determined from image data. For example, video or digital images of a cervical cellular sample are captured using a microscope with image capturing capabilities and cell volume is determined from these images using a computerized image analysis system. See, e.g., Drewnowska, K. et al. (1991) Am J Physiol Cell Physiol 260:C121-C131.

Mean cell volume, as expressed in femtoliters (fL, or 10⁻¹⁵ L), can be calculated by the following formula:

$$10 \times \frac{( \% of volume of cervical cells in a cervical cellular sample )}{( \begin{matrix}number of cervical cells in \\ cervical cellular sample ( millions \text{/} µl )\end{matrix} )}$$

As supported by the data appearing in the Experimental section below, mean corpuscular volume has predictive value in determining whether a particular cervical cellular sample has been infected with HPV or has been transformed. Therefore, such data can be used in the methods provided herein to determine whether a particular cervical cellular sample should be tested for cervical cancer.

In some instances, the cytological data is nuclear to cytoplasmic ratio (NC ratio) data. As used herein, the phrases “nuclear to cytoplasmic ratio,” “nucleus:cytoplasm ratio,” “nucleus-cytoplasm ratio,” “N:C ratio,” “N/C” and “NC ratio” all refer to the ratio of the size (i.e., volume) of the nucleus of a cell (e.g., a cervical cell) to the size of the cell's cytoplasm. As supported by the data appearing in the Experimental section below, nuclear to cytoplasmic ratio data has predictive value with respect to abnormal cytology and/or transformation of cervical cells. Therefore, such data can be used in the methods provided herein to determine whether a particular cervical cellular sample should be tested for cervical cancer.

Nuclear to cytoplasmic ratio can be assayed by any suitable method. For example, nuclear to cytoplasmic ratio can be determined using fluorescence imaging techniques (e.g., confocal microscopy techniques in combination with an image analyzer). See, e.g., Wang et al. (2010) PLoS Pathog 6(11): e1001186. For example, the cytoplasm of cervical cells from the cervical cellular sample can be fluorescently labeled in the cytoplasmic region with a first fluorescent dye and nuclei of these cells are counterstained with a second fluorescent dye that can be distinguished from the first fluorescent dye. Images of these fluorescently labeled cells are captured and quantification of the nuclear to cytoplasmic ratio of these stained cells can then be performed from the images using densitometric software. Examples of a fluorescent dye that can be used in the methods provided herein include, but are not limited to, Propidium Iodide (PI), Ethidium Bromide, 4′,6-diamindino-2-phenylindole (DAPI) and Hoechst dyes 33342 and 33258, DRAQ5, TOPRO-3, and TOTO-3. (see, e.g., Schmidt et al., 2008, “Visual estimates of nucleus-to-nucleus ratios: can we trust our eyes to use the Bethesda ASCUS and LSIL size criteria?” Cancer 114(5):287-93).

In some embodiments, the cytological data is cell cycle data. As used herein, cell cycle data refers to data relating to the cell cycle stage (e.g., G₁, S, G₂, M) of a cell or cells of the cervical cellular sample that is being assayed in the methods provided herein. As supported by the data in the Experimental section below, cell cycle data has predictive value with respect to HPV infection, abnormal cytology and transformation of cervical cells. Therefore, such data can be used in the methods provided herein to determine whether a particular cervical cellular sample should be tested for cervical cancer.

Cell cycle data may be assayed by any suitable method. For example, cell cycle data may be assayed by staining the cervical cell sample with a dye that is capable of labeling the DNA of the cells of the cervical cell sample in a stoichiometric manner (the amount of labeling is directly proportional to the amount of DNA) and subsequent analysis of the labeled sample. In certain embodiments, the labeled sample is analyzed using a device that can determine cell cycle data based on the labeling. Any suitable dye that is capable of labeling DNA and being detected can be used. In some embodiments, the dye is a fluorescent dye. Examples of a fluorescent dye that can be used in the methods provided herein include, but are not limited to, Propidium Iodide (PI), Ethidium Bromide, Hoechst 33342 (2′-[4-ethoxyphenyl]-5-[4-methyl-1-piperazinyl]-2,5′-bi-1H-benzimidazole) and Hoechst 33258 (2′-[4-ethoxyphenyl]-5-[4-methyl-1-piperazinyl]-2,5′-bi-1H-benzimidazole) and others of the Hoechst series; SYTO 40, SYTO 11, 12, 13, 14, 15, 16, 20, 21, 22, 23, 24, 25 (green); SYTO 17, 59 (red), DAPI, DRAQ5™ (an anthraquinone dye with high affinity for double stranded DNA), YOYO-1, propidium iodide, YO-PRO-3, TO-PRO-3, YOYO-3 and TOTO-3, SYTOX Green, SYTOX, methyl green, acridine homodimer, 7-aminoactinomycin D, 9-amino-6-chloro-2-methoxyactridine.

In certain embodiments, the cell cycle data is the percentage of cells in a cervical cellular sample that are at a particular cell cycle stage (e.g., G₁ percentage data, S percentage data, G₂ percentage data, M percentage data). In some embodiments, the cell cycle data is the ratio of cells in a cervical cellular sample that are in a particular cell cycle stage to cells in the cervical sample that are in another cell cycle stage (e.g., G₁/S, G₁/G₂, G₁/M, G₂/G₁, G₂/S, S/G₁, S/G₂, and S/M ratio). In other embodiments, the cell cycle data is the percentage of cells in the cervical cellular sample that have not entered a particular cell cycle stage (e.g., pre G₁ percentage data, pre S percentage data, pre G₂ percentage data, pre M percentage data). In yet other embodiments, the cell cycle data is the percentage of cells in the cervical cellular sample that have already undergone a particular stage of the cell cycle (e.g., post G₁ percentage data, post S percentage data, post G₂ percentage data, post M percentage).

In specific embodiments, the cytological data is post G₁ percentage data. “Post G₁ percentage data” with respect to the cervical cellular sample provided herein, refers to the percentage of cells in the sample that have already undergone the G₁ cell cycle stage. Post G₁ percentage data can be determined by any suitable method. For example, post G₁ percentage data, can be determined by labeling the DNA of a cervical cellular sample with a DNA labeling dye and analyzing the percentage of post G₁ cell in the sample based on the labeled DNA using an automated analyzer, for example, a flow cytometer. See, e.g., Darzynkiewicz, Z. et al. (2004) Cytometry Part A 58A:21-32. As supported by the data appearing in the Experimental section below, post G₁ percentage data has predictive value with respect to abnormal cytology and/or transformation of cervical cells. Therefore, such data can be used in the methods provided herein to determine whether a particular cervical cellular sample should be tested for cervical cancer.

Another aspect of the method is a step of determining from the cytological data whether the sample should be tested for cervical cancer. As discussed above, cytological data (e.g., MCV data, NC ratio data, post G₁ data) has predictive value with respect to cervical cellular transformation and/or abnormal morphology. Accordingly, cytological data acquired from a cervical cellular sample in the assaying step of the subject method can be used to determine whether the sample should be tested for cervical cancer.

The determining step can be performed by a person, for example, one who is knowledgeable about whether a particular value of cytological data acquired for a cervical cellular sample is indicative of transformation and/or abnormal morphology and, therefore, if the sample should be tested for cervical cancer. Alternatively, the determining step can be performed by a processing module, for example, as part of a device described herein or computer.

The determining step can be performed on one, two, three, four, five, six, seven, eight, nine or ten or more different types of cytological data. In certain embodiments, the determining step is performed on one of MCV data, NC ratio data and post G₁. In certain embodiments, the determining step is performed on MCV data. In other embodiments, the determining step is performed on NC ratio data. In yet other embodiments, the determining step is performed on post G₁ data.

In certain embodiments of the method, wherein the cytological data contains MCV data, the cervical cellular sample is determined to be tested for cervical cancer if the average MCV of the cells in the sample is 205 or greater, such as 210 or greater, 215 or greater, 220 or greater, 225 or greater, 230 or greater, 235 or greater, 240 or greater, 245 or greater, 250 or greater, including 260 or greater.

In certain embodiments of the method, wherein the cytological data contains nuclear to cytoplasmic ratio data, the cervical cellular sample is determined to be tested for cervical cancer if the average nuclear to cytoplasmic ratio of the cells in the sample is 0.50 or greater, 0.55 or greater, 0.60 or greater, 0.65 or greater, 0.70 or greater, 0.75 or greater, 0.80 or greater, 0.85 or greater, 0.90 or greater, 1.00 or greater, 1.10 or greater, 1.15 or greater, 1.20 or greater, 1.25 or greater, 1.30 or greater, 1.35 or greater, 1.40 or greater, 1.45 or greater, 1.50 or greater, 1.55 or greater, 1.60 or greater, 1.65 or greater, 1.70 or greater, 1.75 or greater, 1.80 or greater, 1.85 or greater, 1.9 or greater 0, 1.95 or greater, or 2.00 or greater.

In certain embodiments, wherein the cytological data contains post G₁ percentage data, the cervical cellular sample is determined to be tested for cervical cancer if the post G₁ percentage of the sample is 1% or more, 2% or more, 3% or more, 4% or more, 5% or more, 6% or more, 7% or more, 8% or more, 9% or more, 10% or more, 11% or more, 12% or more, 13% or more, 14% or more, 15% or more, 16% or more, 17% or more, 18% or more, 19% or more, 20% or more, 25% or more, 30% or more, 35% or more, 40% or more, 45% or more, 50% or more, 55% or more, 60% or more, 65% or more, 70% or more, 75% or more, 80% or more, 85% or more, 90% or more, 95% or more, 96% or more, 97% or more, 98% or more, or 99% or more. In specific embodiments, the cervical cellular sample should be tested for cervical cancer if the post G₁ percentage of the sample is 3% or greater.

In another aspect of the method, the determining step can be performed within less than 24 hours after the step of assaying the cervical cellular sample to obtain cytological data. In certain embodiments, the determining step is performed less than 23 hours, less than 22 hours, less than 21 hours, less than 20 hours, less than 19 hours, less than 18 hours, less than 17 hours, less than 16 hours less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, less than 1 hour or less than 30 minutes after the assaying step.

In other embodiments, the determining step is performed less than 29 minutes, less than 28 minutes, less than 27 minutes, less than 26 minutes, less than 25 minutes, less than 24 minutes, less than 23 minutes, less than 22 minutes, less than 21 minutes, less than 20 minutes, less than 19 minutes, less than 18 minutes, less than 17 minutes, less than 16 minutes, less than 15 minutes, less than 14 minutes, less than 13 minutes, less than 12 minutes, less than 11 minutes, less than 10 minutes, less than 9.5 minutes, less than 9 minutes, less than 8.5 minutes, less than 8 minutes, less than 7.5 minutes, less than 7 minutes, less than 6.5 minutes, less than 6 minutes, less than 5.5 minutes, less than 5 minutes, less than 4.5 minutes, less than 4 minutes, less than 3.5 minutes, less than 3 minutes, less than 2.5 minutes, less than 2 minutes, less than 1.5 minutes, or less than 1 minute after the assaying step.

In yet other embodiments, the determining step is performed less than 55 seconds, less than 50 seconds, less than 45 seconds, less than 40 seconds less than 35 seconds, less than 30 seconds, less than 25 seconds, less than 20 seconds, less than 15 seconds, less than 10 seconds, less than 9 seconds, less than 8 seconds, less than 7 seconds, less than 6 seconds, less than 5 seconds, less than 4 seconds, less than 3 seconds, less than 2 seconds or less than 1 second after the assaying step.

In certain embodiments, the method for determining whether a cervical cellular sample should be tested for cervical cancer further comprises a step of obtaining the cervical cellular sample from a subject prior to the assaying step. Any suitable protocol for obtaining a cervical cellular sample from a subject may be employed. Examples of protocols of interest include protocols that employ a cervical brush or broom device to collect cells from the surface of the cervix and the endocervix. Descriptions of examples of cervical cell collection devices that may find use in methods provided herein are provided in U.S. Pat. Nos. 2,955,591; 3,626,470; 3,815,580; 3,877,464; 3,881,464; 3,945,372; 4,127,113; 4,175,008; 4,700,713; 4,754,764; 4,762,133; 4,754,764; 4,873,992; 4,862,899; 4,953,560; 5,445,164; 5,787,891; 5,795,309; 6,387,058 and 6,740,049.

Where desired, the obtained cervical cellular sample may be assessed for adequacy prior to proceeding further in the process. For example, an aliquot of the sample may be subjected to light scatter analysis to determine whether adequate target cells are present in the sample, e.g., as described in U.S. Pat. No. 6,329,167; the disclosure of which is herein incorporated by reference.

In some embodiments of the method, the obtained cervical cellular sample is converted to a fluid cervical cellular sample. A fluid cervical cellular sample can be prepared by taking a cervical cellular sample and combining it with a suitable fluid medium. Liquid mediums of interest include, but are not limited to: saline, or balanced salt, solutions (such as Hanks' balanced salt solution, a minimal essential (MEM) tissue culture medium, POLYSAL™ solution, and normal saline); cytology mediums, e.g., Universal Collection Medium (UCM); the universal collection medium described in U.S. Pat. No. 7,371,518 (the disclosure of which is herein incorporated by reference); Standard Transport Medium (STM), PRESERVCYT™ fluid medium (Cytyc, Inc. (Boxborough, Mass.)); CytoRich™ fluid medium (TriPath, Inc. (Burlington, N.C.); and the like.

Following preparation, the resultant fluid cervical cellular sample may be fixed and/or permeabilized as desired. As such, methods provided herein can include fixing the cellular sample by contacting the sample with a suitable fixation reagent. Fixation reagents of interest are those that fix the cells at a desired timepoint. Any convenient fixation reagent may be employed, where suitable fixation reagents include, but are not limited to: formaldehyde, paraformaldehyde, formaldehyde/acetone, methanol/acetone, IncellFP (IncellDx, Inc) etc. For example, paraformaldehyde used at a final concentration of about 1 to 2% has been found to be a good cross-linking fixative. In some instances, the cells in the sample are permeabilized by contacting the cells with a permeabilizing reagent. Permeabilizing reagents of interest are reagents that allow the labeled biomarker probes, e.g., as described in greater detail below, to access to the intracellular environment. Any convenient permeabilizing reagent may be employed, where suitable reagents include, but are not limited to: mild detergents, such as Triton X-100, NP-40, saponin, etc.; methanol, and the like. It may also be desirable to label cells with a positive heavy metal control, e.g. a DNA intercalator labeled with a heavy metal, e.g. iridium, etc. Cells may also be stained with a viability dye prior to fixation, e.g. ethidium bromide, propidium iodide, DAPI, RhCl₃, etc., as desired.

In certain embodiments of the method, the step of assaying the cervical cellular sample to obtain cytological data is carried out at the location of sample obtainment. In other embodiments, the step of determining from the cytological data whether the sample should be tested for cervical cancer is carried out at the location of sample obtainment. In yet other embodiments, the assaying step and the determining step are both carried out at the location of sample obtainment. As used herein, “at the location of sample obtainment” refers to the same room, the same building, the same building complex, same vehicle or at a distance of 200 meters or less, 175 meters or less, 150 meters or less, 125 meters or less, 100 meters or less, 90 meters or less, 85 meters or less, 80 meters or less, 75 meters or less, 70 meters or less, 65 meters or less, 60 meters or less, 55 meters or less, 50 meters or less, 45 meters or less, 40 meters for less, 35 meters or less, 30 meters or less, 25 meters or less, 20 meters or less, 15 meters or less, 14 meters or less, 13 meters or less, 12 meters or less, 11 meters or less, 10 meters or less, 9 meters or less, 8 meters or less, 7 meters or less, 6 meters or less, 5 meters or less, 4 meters or less, 3 meters or less, 2 meters or less, 1 meter or less or less than 1 meter from the location of sample obtainment.

In certain embodiments, the step of assaying the cervical cellular sample to obtain cytological data is carried out at a different location than the location of sample obtainment. In some embodiments, the step of determining from the cytological data whether the sample should be tested for cervical cancer is carried out at a different location than the location of sample obtainment. As used herein, “at a different location than the location of sample obtainment” refers to a different room, a different building, a different building complex, or different vehicle than the location of the sample obtainment or a distance of 200 meters or more, 250 meters or more, 300 meters or more, 350 meters or more, 400 meters or more, 450 meters or more, 500 meters or more, 550 meters or more, 600 meters or more, 650 meters or more, 700 meters or more, 750 meters or more, 800 meters or more, 850 meters or more, 900 meters or more, 950 meters or more, 1 kilometer or more, 5 kilometers or more, 10 kilometers or more, 20 kilometers or more, 30 kilometers or more, 40 kilometers or more, 50 kilometers or more or 100 milometers or more from the location of sample obtainment.

In certain embodiments, the step of assaying the cervical cellular sample to obtain cytological data is carried out at the location of sample obtainment and the step of determining from the cytological data whether the sample should be tested for cervical cancer is carried out at a different location than the location of sample obtainment. For example, a cervical cellular sample may be assayed to obtain cytological data at the location of sample obtainment and the cytological data is then sent by a wired or wireless protocol (e.g., by electronic mail, by fax, by cellular transmission, by satellite) to a different location than the location of sample obtainment, where the step of determining from the cytological data whether the sample should be tested for cervical cancer is carried out.

In some embodiments, the method includes providing a recommendation as to whether a cervical sample should be tested for cervical cancer. In such embodiments, the recommendation may be provided by providing, i.e. generating, a written report that includes an assessment as to whether a cervical sample should be tested for cervical cancer. Thus, the methods provided herein may further include a step of generating or outputting a report providing a recommendation of whether a cervical cellular sample should be tested for cervical cancer, such a report can be provided in the form of an electronic medium (e.g., an electronic display on a device described herein or a computer monitor), or in the form of a tangible medium (e.g., a report printed on paper or other tangible medium).

A “report,” as described herein, is an electronic or tangible document which includes report elements that provide information of interest relating to a subject monitoring assessment and its results. A subject report includes at least a recommendation as to whether a cervical cellular sample should be tested for cervical cancer. A subject report can be completely or partially electronically generated. A subject report can further include one or more of: 1) information regarding the testing facility; 2) service provider information; 3) subject data; 4) sample data; 5) an assessment report, which can include various information including: a) reference values employed, and b) data collected (e.g., MCV data, NC ratio data, and/or post G₁ percentage data); and 6) other features.

The report may include information about the testing facility, which information is relevant to the hospital, clinic, or laboratory in which sample gathering and/or data collection and determination was conducted. Sample gathering can include obtaining a cervical cellular sample from a subject. Data collection/determination can include information regarding the data collected and used to determine whether the cervical cellular sample should be tested for cervical cancer (e.g., MCV data, NC ratio data, and/or post G₁ percentage data). This information can include one or more details relating to, for example, the name and location of the testing facility, the identity of the lab technician who conducted the assay and/or who entered the input data, the date and time the assay was conducted and/or analyzed, the location where the sample and/or result data is stored, the lot number of the reagents (e.g., kit, etc.) used in the assay, and the like. Report fields with this information can generally be populated using information provided by the person performing the method.

The report may include information about the service provider, which may be located outside the healthcare facility at which the user is located, or within the healthcare facility. Examples of such information can include the name and location of the service provider, the name of the reviewer, and where necessary or desired the name of the individual who conducted sample collection and/or data generation. Report fields with this information can generally be populated using data entered by the user, which can be selected from among pre-scripted selections (e.g., using a drop-down menu). Other service provider information in the report can include contact information for technical information about the result and/or about the interpretive report.

The report may include data section regarding the subject whom the cervical cellular sample was obtained, including subject's medical history (which can include, e.g., age, race, serotype, prior preeclampsia episodes, and any other characteristics of the pregnancy), as well as administrative subject data such as information to identify the subject (e.g., name, patient date of birth (DOB), gender, mailing and/or residence address, medical record number (MRN), room and/or bed number in a healthcare facility), insurance information, and the like), the name of the subjects's physician or other health professional who ordered the monitoring assessment and, if different from the ordering physician, the name of a staff physician who is responsible for the subject's care (e.g., primary care physician).

The report may include a sample data section, which may provide information about the cervical cellular sample analyzed, such as how the sample was handled (e.g. storage temperature, preparatory protocols) and the date and time collected. Report fields with this information can generally be populated using data entered by the user, some of which may be provided as pre-scripted selections (e.g., using a drop-down menu).

It will also be readily appreciated that the reports can include additional elements or modified elements. For example, where electronic, the report can contain hyperlinks which point to internal or external databases which provide more detailed information about selected elements of the report. For example, the patient data element of the report can include a hyperlink to an electronic patient record, or a site for accessing such a patient record, which patient record is maintained in a confidential database. This latter embodiment may be of interest in an in-hospital system or in-clinic setting. When in electronic format, the report is recorded on a suitable physical medium, such as a computer readable medium, e.g., in a computer memory, zip drive, CD, DVD, etc.

It will be readily appreciated that the report can include all or some of the elements above, with the proviso that the report generally includes at least the elements sufficient to provide a recommendation as to whether a cervical cellular sample should be tested for cervical cancer.

In certain embodiments, aspects of the methods further comprise a step of outputting a report that includes a recommendation as to whether a sample should be tested for cervical cancer based on the step of determining from the cytological data whether the sample should be tested for cervical cancer.

In certain embodiments, the outputting step is performed within less than 24 hours after the determining step. In specific embodiments, the outputting step is performed less than 23 hours, less than 22 hours, less than 21 hours, less than 20 hours, less than 19 hours, less than 18 hours, less than 17 hours, less than 16 hours less than 15 hours, less than 14 hours, less than 13 hours, less than 12 hours, less than 11 hours, less than 10 hours, less than 9 hours, less than 8 hours, less than 7 hours, less than 6 hours, less than 5 hours, less than 4 hours, less than 3 hours, less than 2 hours, less than 1 hour or less than 30 minutes after the determining step.

In specific embodiments, the outputting step is performed less than 29 minutes, less than 28 minutes, less than 27 minutes, less than 26 minutes, less than 25 minutes, less than 24 minutes, less than 23 minutes, less than 22 minutes, less than 21 minutes, less than 20 minutes, less than 19 minutes, less than 18 minutes, less than 17 minutes, less than 16 minutes, less than 15 minutes, less than 14 minutes, less than 13 minutes, less than 12 minutes, less than 11 minutes, less than 10 minutes, less than 9.5 minutes, less than 9 minutes, less than 8.5 minutes, less than 8 minutes, less than 7.5 minutes, less than 7 minutes, less than 6.5 minutes, less than 6 minutes, less than 5.5 minutes, less than 5 minutes, less than 4.5 minutes, less than 4 minutes, less than 3.5 minutes, less than 3 minutes, less than 2.5 minutes, less than 2 minutes, less than 1.5 minutes, or less than 1 minute after the determining step.

In yet other embodiments, the outputting step is performed less than 55 seconds, less than 50 seconds, less than 45 seconds, less than 40 seconds less than 35 seconds, less than 30 seconds, less than 25 seconds, less than 20 seconds, less than 15 seconds, less than 10 seconds, less than 9 seconds, less than 8 seconds, less than 7 seconds, less than 6 seconds, less than 5 seconds, less than 4 seconds, less than 3 seconds, less than 2 seconds or less than 1 second after the determining step.

In some embodiments, the recommendation is outputted at the location of sample obtainment. In other embodiments, the recommendation is outputted at a location different than the location of sample of obtainment and is then sent by a wired or wireless protocol (e.g., by electronic mail, by fax, by cellular transmission, by satellite) to the location of sample obtainment.

In certain embodiments, the method further comprises a step of forwarding the cervical cellular sample to a cervical cancer testing facility if a determination is made that the cervical cellular sample should be tested for cervical cancer. In some embodiments, the same aliquot of the cervical cellular sample that is used in the assaying and determining step is forwarded to the cervical cancer testing facility. In other embodiments, an aliquot of the cervical cellular sample that is different than the aliquot used in the assaying and determining step is forwarded to the cervical cancer testing facility. A cervical cellular sample this is forwarded to a cervical cancer testing facility can then be tested for cervical cancer by using any suitable method, for example, using a Pap test (e.g., ThinPrep®, Linder et al (1998) Archives of Pathology & Laboratory Medicine 122(2): 139-144; Abulafia et al. (2003) Oncology 90: 137-144) and/or an HPV test (e.g., Hybrid Capture® or PCR, Poljak et al. (2002) J Clin Virol 25(Supp 3): 89-97; Clavel et al. J Clin Pathol (1998) 51:737-740; Rozendaal et al. (1996) Int J Cancer 68(6): 766-769). In particular embodiments, the cervical cellular sample is tested at the cervical cancer testing facility using a method based on morphometric data as well as biomarker data (e.g., HPV genes L1, L2, E2, E4, E5, E6 or E7) and/or non-specific cell data. See, e.g., U.S. Pat. Nos. 7,524,631; 7,888,032, and US Patent Publication No. 2012/0122078A1.

### Devices and Systems

In another aspect, provided is a device for use in practicing the methods provided herein. Such devices, for example, may allow for the collection of data from a cervical cellular sample that is predictive of abnormal cytology, transformation, and/or HPV infection. In certain embodiments, the device includes a processing module that can determine from the collected data whether a sample should be tested for cervical cancer.

In certain embodiments, the device includes a data collector configured to obtain cytological data from a cervical cellular sample; and a processing module configured to receive cytological data from the collector and determine from the cytological data whether the sample should be tested for cervical cancer. In certain embodiments, the device outputs a recommendation based on the determination made by the processing module as to whether the cervical cellular sample should be tested for cervical cancer.

The data collector may be configured for obtaining any of the cytological data discussed herein. The data collector may be configured to obtain one, two, three, four, five, six, seven, eight, nine or ten or more different types of cytological data. In certain embodiments, the data collector is configured for obtaining morphological data (e.g., mean corpuscular volume (MCV) data or nuclear to cytoplasmic ratio (NC ratio) data). In particular embodiments, the data collector is configured for obtaining MCV data. In particular embodiments, the data collector is configured to obtain NC ratio data. In other embodiments, the data collector is configured to obtain cell cycle data. In specific embodiments, the cell cycle data is post G₁ percent data. In certain embodiments, the data collector comprises a light detector.

In certain embodiments the device includes a sample holder that is operatively coupled to the data collector. The sample holder is configured in a manner as to allow the data collector to obtain sufficient cytological data to carry out the methods as described herein. For example, when the sample is a liquid based cervical cellular sample, the sample holder is configured to hold a sufficient amount of the liquid cervical cellular sample for the data collector to obtain sufficient cytological data (e.g., MCV data, NC ratio data, post G₁ percentage data) to carry out the methods as described herein. Alternatively, when the sample is affixed to a slide, the sample holder is configured in a manner so that the slide is held in place (e.g., by clips or a fastener) to allow for the data collector to obtain sufficient cytological data. The sample holder can be made of any material that allows for the data collector to obtain cytological data. In specific embodiments, the sample holder is made of a material that is transparent to the types of illumination used or is present in the methods described herein.

Aspects of the invention further include a variety of computer-related embodiments. Specifically, the step of determining from the cytological data whether a cervical cellular sample should be tested for cervical cancer described in previous sections may be performed using a computer-based system or processing module.

A “computer-based system” refers to the hardware, software, and data storage used to analyze the information of the present invention. The minimum hardware of the computer-based systems of the present invention comprises a central processing unit (CPU) or processing module, input means, output means, and data storage means. A skilled artisan can readily appreciate that any one of the currently available computer-based system are suitable for use in the present invention. The data storage means may comprise any manufacture comprising a recording of the present information as described above, or a memory access means that can access such a manufacture. In certain embodiments, the computer based system is integrated into a device as described herein.

In another aspect, provided herein is a processing module configured to receive cytological data from the data collector and determine whether the cervical cellular sample should be tested for cervical cancer. A “processing module” or “processor” references any hardware and/or software combination that will perform the functions required of it. For example, any processor herein may be a programmable digital microprocessor such as available in the form of an electronic controller, mainframe, server or personal computer (desktop or portable).

In specific embodiments, the processing module is programmed to receive data collected from a data collector and use the data to determine whether a cervical cellular sample should be tested for cervical cancer. In specific embodiments, the processing module is integrated into a device as provided herein.

In certain embodiments, instructions for receiving data collected from a data collector and determining whether a cervical cellular sample should be tested for cervical cancer based on the collected data are coded onto a physical computer-readable medium in the form of “programming”, where the term “computer readable medium” as used herein refers to any storage or transmission medium that participates in providing instructions and/or data to a processing module for processing. In certain embodiments, the device provided herein includes a computer-readable medium. In particular embodiments, the computer-readable medium includes a program code for receiving data collected from a data collector and using the data to determine whether a cervical cellular sample should be tested for cervical cancer. In certain embodiments, the computer-readable medium includes a program code for outputting a recommendation as to whether a cervical cellular sample should be tested for cervical cancer based on a determination made by the processing module. The program code, when executed by the processing module, causes the processing module to perform functions described herein. In other embodiments, some functions are implemented primarily in hardware using, for example, a hardware state machine. Implementation of the hardware state machine so as to perform the functions described herein may be accomplished using any suitable method.

Examples of computer readable media include, but are not limited to, floppy disks, magnetic tape, CD-ROM, a hard disk drive, a ROM or integrated circuit, a magneto-optical disk, or a computer readable card such as a PCMCIA card, a portable flash drive, and the like, whether or not such devices are internal or external to the computer. A file containing information may be “stored” on computer readable medium, where “storing” means recording information such that it is accessible and retrievable at a later date by a computer. Of interest as media are non-transitory media, i.e., physical media in which the programming is associated with, such as recorded onto, a physical structure. Non-transitory media does not include electronic signals in transit via a wireless protocol.

In certain embodiments, the methods are coded and stored in a non-volatile computer-readable medium such as ROM, EPROM or flash memory. Such memory devices may, in turn, be incorporated as part of a processing module of a device provided herein.

With respect to computer readable media, “permanent memory” refers to memory that is permanent. Permanent memory is not erased by termination of the electrical supply to a computer or processor. Computer hard-drive, CD-ROM, floppy disk and DVD are all examples of permanent memory. Random Access Memory (RAM) is an example of non-permanent memory. A file in permanent memory may be editable and re-writable.

In embodiments where the processing module is programmable, suitable programming can be communicated from a remote location to the processing module, or previously saved in a computer program product (such as a portable or fixed computer readable storage medium, whether magnetic, optical or solid state device based). For example, a magnetic medium or optical disk may carry the programming, and can be read by a suitable reader communicating with each processor at its corresponding station.

To “record” data, programming or other information on a computer readable medium refers to a process for storing information, using any such methods as known in the art. Any convenient data storage structure may be chosen, based on the means used to access the stored information. A variety of data processor programs and formats can be used for storage, e.g. word processing text file, database format, etc.

In certain embodiments, the processing module is integrated into a device provided herein. In other embodiments, the processing module is distributed from the device where the processing module and device are in communication with each other, e.g., via a wired or wireless communication protocol. In such embodiments, the processing module may send a signal to the device once a determination is made as to whether the cervical cellular sample should be tested for cervical cancer and the device renders an output based on the signal from the processing module.

In specific embodiments, the processing module outputs a recommendation as to whether a cervical cellular sample should be tested for cervical cancer after determining whether the cervical cellular sample should be tested for cervical cancer. The recommendation that is output can then be displayed to a user of the device, for example, using a display. In certain embodiments, the device may further include a display (e.g., an LCD screen) that displays to a user of the device the output rendered by the processing module.

In certain embodiments, the device further includes a communications module for facilitating information (e.g., data obtained from the cervical cellular sample, a recommendation as to whether the sample should be tested for cervical cancer) transfer between the device and one or more users.

In certain embodiments, the device is a tabletop or benchtop device that is configured to obtain cytological data from a cervical cellular sample and determine from the data whether the sample should be tested for cervical cancer, e.g., as described above. By tabletop or benchtop device is meant a device having a length ranging from 0.20 m to 1.50 m, such as 0.40 m to 1.25 m, including 0.50 m to 1.0 m, a height ranging from 0.10 m to 1.0 m, such as 0.2 m to 0.8 m and including 0.25 m to 0.75 m and a width ranging from 0.10 m to 0.80 m, such as 0.15 m to 0.75 m and including 0.20 m to 0.70 m. In certain embodiments, the device is configured to occupy a space of 0.002 m³ to 1.20 m³, such as 0.005 m³ to 1.15 m³, and including 0.10 m³ to 1.00 m³, 0.30 m³ to 0.80 m³, and 0.40 m³ to 0.75 m³. While the weight of such a device may vary, in some instances the weight will range from 5.00 kg to 100.00 kg, such as 10.00 kg to 75.00 kg and including 15.00 kg to 50.00 kg. Such configurations allow the device to be stored and operated on a table or bench top, for example, at the location of sample obtainment.

In certain embodiments, the device is a tabletop or benchtop device and includes a data collector capable of collecting at least one of the cytological data provided herein from a cervical cellular sample and a processing module for determining from the collected data whether the sample should be tested for cervical cancer. In specific embodiments, the device is tabletop or benchtop device and includes a data collector capable of collecting at least one of MCV data, NC ratio data and/or post G₁ data from a cervical cellular sample and a processing module for determining from the collected data whether the sample should be tested for cervical cancer.

In certain embodiments, the device is a tabletop or benchtop device and includes a data collector capable of collecting MCV data from a cervical cellular sample and a processing module for determining from the collected data whether the sample should be tested for cervical cancer. Devices that are configured to collect and analyze MCV data may include, for example, a light source capable of illuminating the cells of the sample to produce refracted, diffracted or scatter light and a data collector that includes a light detector, capable of detecting the refracted, diffracted or scatter light (i.e., the MCV data). In another embodiment, the data collector includes a camera capable of capturing and storing images of the cervical cellular sample (i.e., the MCV data). In yet other embodiments, the device includes a channel that allows a cervical cellular sample in an electrolyte solution to pass through and causes a change in electrical resistance and a data collector capable of detecting and measuring the change in electrical resistance (i.e., the MCV data). In embodiments of the device capable of collecting MCV data, the device includes a processing module that can determine from the MCV data whether a sample should be tested for cervical cancer and that is operatively connected to the data collector. Such a processing module may include, for example, coded instructions for determining the MCV of the sample based on the MCV data collected as well as instructions for determining whether a cervical cellular sample should be tested for cervical cancer based on the MCV data. In certain embodiments, the coded instructions are stored on a computer readable medium. In specific embodiments, the device includes a display for displaying a recommendation as to whether a sample should be tested for cervical cancer based on the MCV ratio data, as determined by the processing module. In specific embodiments, the device includes a sample holder for holding the sample during MCV data collection (e.g., a holder configured to hold a sample affixed to a slide or a volume of a liquid based sample).

In certain embodiments, the device is a tabletop or benchtop device and includes a data collector capable of collecting NC ratio data from a cervical cellular sample and a processing module for determining from the collected data whether the sample should be tested for cervical cancer. Devices that are configured to collect and analyze NC ratio data may include, for example, a camera capable of capturing and storing images (e.g. images of fluorescently labeled samples) of the cervical cellular sample (i.e., the NC ratio data). In embodiments of the device capable of collecting NC ratio data, the device includes a processing module that can determine from the NC ratio data whether a sample should be tested for cervical cancer and that is operatively connected to the data collector. Such a processing module may include coded instructions for determining the NC ratio of the sample based on the NC ratio data collected (e.g. image analysis/densitometric software) as well as coded instructions for determining whether a cervical cellular sample should be tested for cervical cancer based on the determined NC ratio. In certain embodiments, the coded instructions are stored on a computer readable medium. In specific embodiments, the device includes a display for displaying a recommendation as to whether a sample should be tested for cervical cancer based on the NC ratio data, as determined by the processing module. In specific embodiments, the device includes a sample holder for holding the sample during NC ratio data collection (e.g., a holder configured to hold a sample affixed to a slide or a volume of a liquid based sample).

In other embodiments, the device is a tabletop or benchtop device and includes a data collector capable of collecting post G₁ percentage data from a cervical cellular sample and a processing module for determining from the collected data whether the sample should be tested for cervical cancer, respectively. Devices that are configured to collect and analyze post G₁ percentage data may include, for example, a camera capable of capturing and storing images (e.g. images of fluorescently labeled samples) of the cervical cellular sample. In other embodiments, the device includes a light source capable of illuminating the cells of the sample to produce refracted, diffracted or scatter light and a data collector that includes a light detector, capable of detecting the refracted, diffracted or scatter light. In certain embodiments, the device includes a processing module that can determine from the G₁ percentage data whether a sample should be tested for cervical cancer and that is operatively connected to the data collector. Such a processing module may include coded instructions for determining the post G₁ percentage of the sample based on the post G₁ percentage data collected (e.g. image analysis/densitometric software) as well as coded instructions for determining whether a cervical cellular sample should be tested for cervical cancer based on the determined post G₁ percentage. In certain embodiments, the coded instructions are stored on a computer readable medium. In specific embodiments, the device includes a display for displaying a recommendation as to whether a sample should be tested for cervical cancer based on the post G₁ percentage data, as determined by the processing module. In specific embodiments, the device includes a sample holder for holding the sample during post G₁ percentage data collection (e.g., a holder configured to hold a sample affixed to a slide or a volume of a liquid based sample).

In other embodiments, the device is a tabletop or benchtop device and includes a data collector capable of collecting MCV data, NC ratio data, and post G₁ percentage data from a cervical cellular sample and a processing module for determining from one or more of the collected data whether the sample should be tested for cervical cancer, respectively. Devices that are configured to collect and analyze MCV data, NC ratio data, and post G₁ percentage data may include, for example, a light source capable of illuminating the cells of the sample to produce refracted, diffracted or scatter light and a data collector that includes a light detector, capable of detecting the refracted, diffracted or scatter light. In some embodiment, the data collector includes a camera capable of capturing and storing images of the cervical cellular sample. In yet other embodiments, the device includes a channel that allows a cervical cellular sample in an electrolyte solution to pass through and causes a change in electrical resistance and a data collector capable of detecting and measuring the change in electrical resistance. In certain embodiments, the data collector is configured to collect more than one type of the cytological data described herein. For example, the data collector may include a camera that is capable of capturing images from which NC ratio data and post G₁ percentage is derived from. In embodiments of the device capable of collecting MCV data, NC ratio data, and post G₁ percentage data, the device includes a processing module that can determine from one or more of the MCV data, NC ratio data, and post G₁ percentage whether a sample should be tested for cervical cancer and that is operatively connected to the data collector. Such a processing module may include, for example, coded instructions for determining MCV, NC ratio, and/or post G₁ percentage of the sample based on the data collected as well as instructions for determining whether a cervical cellular sample should be tested for cervical cancer based on the data. In certain embodiments, the coded instructions are stored on a computer readable medium. In specific embodiments, the device includes a display for displaying a recommendation as to whether a sample should be tested for cervical cancer based on the MCV, NC ratio and/or post G₁ percentage data, as determined by the processing module. In specific embodiments, the device includes a sample holder for holding the sample during MCV, NC ratio and/or post G₁ percentage data collection (e.g., a holder configured to hold a sample affixed to a slide or a volume of a liquid based sample).

### Utility

The subject methods, devices, and systems find use in a variety of different applications where determination as to whether a cervical cellular sample should be test for cervical cancer is desired. Particular embodiments provided herein allow for the determination of whether a cervical cellular sample should undergo testing for cervical cancer at the location of sample obtainment. Moreover, in certain embodiments, a recommendation as to whether cervical cellular sample should undergo testing is outputted faster (e.g., one day or less) than current methods used in cervical cancel screening, for example, the Pap test and HPV test discussed above. Further, the subject methods can be performed using an automated analyzer. Such methods, therefore, do not require the highly trained personnel used in traditional Pap and HPV tests. Thus, the subject methods and systems can provide for a convenient, point of care first pass screen that alleviates unnecessary downstream cost-, labor-, and time-intensive testing in certain instances. Moreover, the subject methods and systems utilize data (e.g., NC ratio data, MCV data, post G₁ data) that traditional HPV and Pap tests do not employ. As such, the subject methods and systems can bolster and improve the accuracy of current cervical cancer screening in general.

### Kits

In another aspect, provided herein are kits for practicing the subject methods, e.g., as described above. Kits may include a cytological labeling reagent, e.g. as described above. Kits may also include a sample holder configured to hold a cervical cellular sample that has been labeled with the cytological labeling reagent. Such a device may include a data collector that is configured to obtain the cytological data from the cervical cellular sample and a processing module configured to receive cytological data from the data collector and render an output based on the cytological data as to whether the cervical cellular sample should be tested for cervical cancer.

In addition to the above components, the subject kits will further include instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, etc. Yet another means would be a computer readable medium, e.g., diskette, CD, etc., on which the information has been recorded. Yet another means that may be present is a website address which may be used via the internet to access the information at a removed site. Any convenient means may be present in the kits.

The following examples are offered by way of illustration and not by way of limitation.

### Experimental

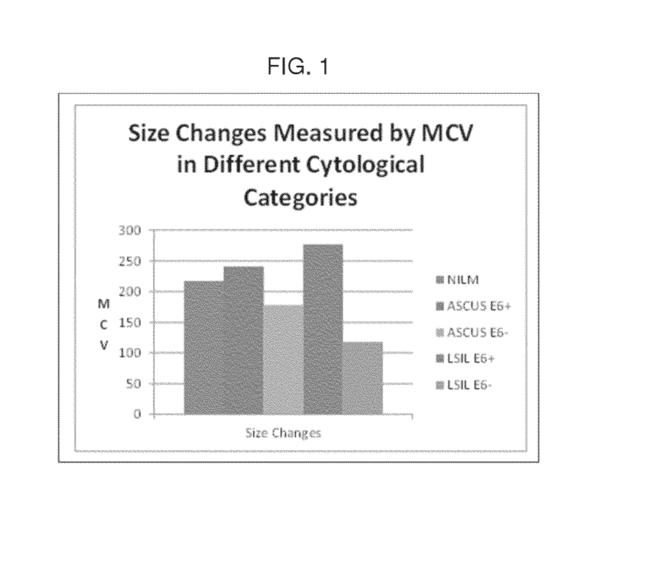

As shown in FIG. 1, HPV infection and/or transformation of squamous cells can cause morphologic changes in these cells that can be determined by mean corpuscular volume (MCV). MCV can be determined using the following formula:

$$10 \times \frac{( \% of volume of cervical cells in a cervical cellular sample )}{( \begin{matrix}number of cervical cells in \\ cervical cellular sample ( millions \text{/} µl )\end{matrix} )}$$

MCV was determined for three different cytological categories of cervical squamous cells: 1) negative for intraepithelial lesion or malignancy (NILM); 2) atypical squamous cells of undetermined significance (ASCUS); and 3) low-grade squamous intraepithelial lesion (LSIL). ASCUS and LSIL squamous cells were further divided into cells that were either positive or negative for E6, an HPV protein that is associated with cervical cancer, and MCV for each of these groups were determined separately.

As shown in FIG. 1, distinct differences were observed in MCV between the different categories of squamous cells analyzed. In particular, ASCUS and LSIL samples that were E6 positive exhibited greater MCV as compared to E6 negative counterparts and NILM samples. As such, MCV can have independent predictive power in determining whether a particular cervical cellular sample should be further tested for cervical cancer.

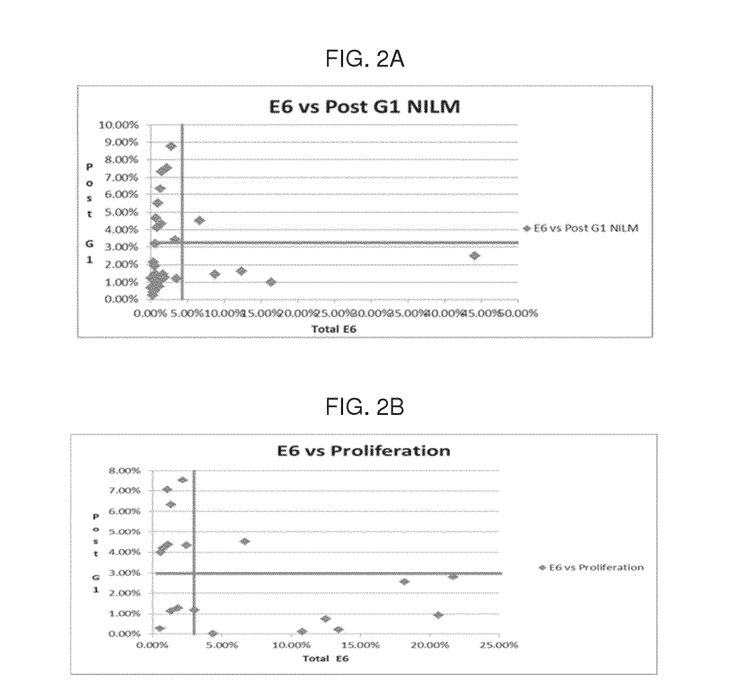

Cell cycle analysis, in particular, post G₁ percentage data, can also be used to independently determine whether a cervical cellular sample should be tested for cervical cancer. FIG. 2 shows the relationship of percentage of cells in cellular samples (FIG. 2A, normal cytology, FIG. 2B, abnormal cytology) that express the cervical cancer cell marker E6 and the percentage of cells that are post G₁. As shown in FIG. 2, a cervical cellular sample that has a post-G1 percent greater than 3% has a lower chance of being abnormal or transformed at the molecular level. NC ratio can be determined by using only one staining reagent, i.e., a non-specific DNA staining reagent.

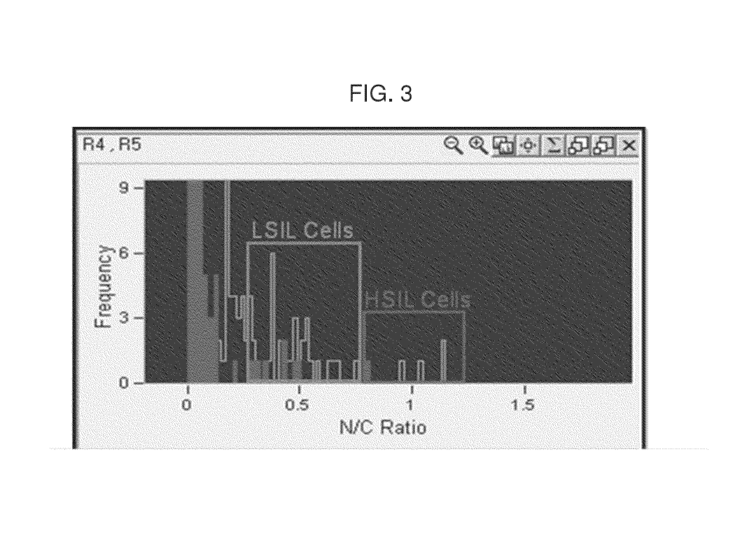

In addition, nuclear to cytoplasmic ratio (NC ratio) is another independent variable that can be used to determine whether a cervical cellular sample should be tested for cervical cancer. As shown in FIG. 3, high-grade squamous intraepithelial lesion (HSIL) cells exhibit greater NC ratio than low-grade squamous intraepithelial lesion (LSIL). As such, alterations in nuclear to cytoplasmic ratios can indicate cytologic abnormalities related to HPV infection.

Although the foregoing invention has been described in some detail by way of illustration and example for purposes of clarity of understanding, it is readily apparent to those of ordinary skill in the art in light of the teachings of this invention that certain changes and modifications may be made thereto without departing from the spirit or scope of the appended claims.

Accordingly, the preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof. Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of present invention is embodied by the appended claims.

1. A method of determining whether a cervical cellular sample should be tested for cervical cancer, the method comprising:
assaying a cervical cellular sample to obtain cytological data; and
determining from the cytological data whether the sample should be tested for cervical cancer.

2. The method according to claim 1, wherein the cervical cellular sample is tested for cytological data at the location of sample obtainment.

3. The method according to claim 1, wherein the cytological data is morphological data.

4. The method according to claim 3, wherein the morphological data is mean corpuscular volume (MCV) data.

5. The method according to claim 3, wherein the morphological data is nuclear to cytoplasmic ratio (NC ratio) data.

6. The method according to claim 1, wherein the cytological data is cell-cycle data.

7. The method according to claim 1, wherein the cell-cycle data is post G₁ percentage data.

8. The method according to claim 1, wherein the cervical cellular sample is a liquid based cytology specimen.

9. The method according to claim 1, wherein assaying a cervical cellular sample comprises labeling the sample with a fluorescent dye.

10. The method according to claim 9, wherein the fluorescent dye is 4′,6-diamindino-2-phenylindole (DAPI).

11. The method according to claim 1, further comprising forwarding the cervical cellular sample to a cervical cancer testing facility if a determination is made that the cervical cellular sample should be tested for cervical cancer.

12-20. (canceled)

21. A device for determining whether a cervical cellular sample should be tested for cervical cancer, the device comprising:
a data collector configured to obtain cytological data from a cervical cellular sample; and
a processing module configured to receive cytological data from the collector and output a result based on data that is a recommendation as to whether the cervical cellular sample should be tested for cervical cancer.

22. The device according to claim 21, wherein the data collector comprises a light detector.

23. The device according to claim 21, wherein the data collector comprises a camera.

24. The device according to claim 21, wherein the device is a tabletop device.

25. The device according to claim 21, wherein the cytological data is morphological data.

26. The device according to claim 25, wherein the morphological data is mean corpuscular volume (MCV) data.

27. The device according to claim 25, wherein the morphological data is nuclear to cytoplasmic ratio (NC ratio) data.

28. The device according to claim 21, wherein the cytological data is cell-cycle data.

29. The device according to claim 21, wherein the cell-cycle data is post G₁ percent data.

30. The device according to claim 21, wherein the device comprises a sample holder operatively coupled to the data collector.

31. The device according to claim 30, wherein the sample holder comprises a cervical cellular sample.

32. A kit comprising:
a cytological labeling reagent;
a sample holder configured to hold a cervical cellular sample that has been labeled with the cytological labeling reagent; wherein the holder is further configured to be operatively coupled to a data collector in a device comprising:
the data collector, wherein the data collector is configured to obtain cytological data from a cervical cellular sample; and
a processing module configured to receive cytological data from the collector and output a result based on data that is a recommendation as to whether the cervical cellular sample should be tested for cervical cancer.
